(19) United States
(12) Patent Application Publication
(10) Pub. No.: US 2018/0228858 A1
(43) Pub. Date: Aug. 16, 2018

(54) A PLANT EXTRACT AND COMPOUNDS FOR USE IN WOUND HEALING

(71) Applicant: Phynova Limited, Long Hanborough (GB)

(72) Inventors: Andrew B. Gallagher, Kidlington (GB); Rolf W. Hartmann, Saarbrücken (DE); Matthias Engel, Homburg (DE); Axel Koch, Saarbrücken (DE); Chris J. Van Koppen, Kleve (DE)

(21) Appl. No.: 15/533,009

(22) PCT Filed: Nov. 30, 2015
(86) PCT No.: PCT/GB2015/000312

§ 371 Date: Jun. 2, 2017

(30) Foreign Application Priority Data
Dec. 3, 2014 (GB) 1421479.5

(51) Int. Cl. A61K 36/537; A61K 47/69; A61K 8/9789; A61Q 11/00

(52) U.S. Cl. CPC A61K 36/537; A61Q 11/00; A61K 8/9789; A61K 47/6955

(57) ABSTRACT

A plant extract, derived from a Salvia spp, may include one of at least one tanshinone compound, or at least one tanshinone compounds including a CYP11B1 inhibitory amount of at least one of tanshinone I and dihydrotanshinone. The plant extract may be used for use in the treatment of a wound or Cushing's syndrome.

This invention relates to a plant extract, derived from a Salvia spp, comprising one or more tanshinone compounds, or said one or more tanshinone compounds, for use in the treatment of wounds, particularly chronic wounds, or other conditions benefiting from inhibition of cortisol production, particularly Cushing's syndrome.

Preferred tanshinone compounds include, but are not limited to, dihydrotanshinone (particularly 15,16-dihydrotanshinone (CAS No. 87205-99-0)) and Tanshinone I.

Preferred treatments include the treatment of chronic wounds (generally defined as wounds that take longer than 6 weeks to heal). Such wounds are particularly common in obese patients and those suffering from diabetes, as well as in bed-ridden patients (decubitus or bedsores) and patients who have undergone external beam radiation therapy.

A chronic wound does not heal in an orderly set of stages and in a predictable amount of time the way most wounds do. Chronic wounds seem to be detained in one or more of the phases of wound healing. In contrast, in acute wounds, there is a precise balance between production and degradation of molecules such as collagen; in chronic wounds this balance is lost and degradation plays too large a role.

Chronic wounds may never heal or may take years to do so. These wounds cause patients severe emotional and physical stress and create a significant financial burden on patients and the whole healthcare system.

Acute and chronic wounds are at opposite ends of a spectrum of wound healing types that progress toward being healed at different rates.

The vast majority of chronic wounds can be classified into three categories: venous ulcers, diabetic, and pressure ulcers. A small number of wounds that do not fall into these categories may be due to causes such as radiation or ischemia.

Venous ulcers, which usually occur in the legs, account for about 70% to 90% of chronic wounds-and mostly affect the elderly. They are thought to be due to venous hypertension caused by improper function of valves that exist in the veins to prevent blood from flowing backward. Ischemia results from the dysfunction and, combined with reperfusion injury, causes the tissue damage that leads to the wounds.

Diabetic ulcers are another major cause of chronic wounds. Diabetics have a 15% higher risk of amputation than the general population due to chronic ulcers. Diabetes causes neuropathy, which inhibits nociception and the perception of pain. Thus patients may not initially notice small wounds to legs and feet, and may therefore fail to prevent infection or repeated injury. Further, diabetes causes immune compromise and damage to small blood vessels, preventing adequate oxygenation of tissue, which can cause chronic wounds. Pressure also plays a role in the formation of diabetic ulcers.

Pressure ulcers which usually occur in people with conditions such as paralysis that inhibits movement of body parts that are commonly subjected to pressure such as the heels, shoulder blades, and sacrum. Pressure ulcers are caused by ischemia that occurs when pressure on the tissue is greater than the pressure in capillaries, and thus restricts blood flow into the area. Muscle tissue, which needs more oxygen and nutrients than skin does, shows the worst effects from prolonged pressure. As in other chronic ulcers, reperfusion injury damages tissue.

The extracts and active compounds of the formulation may be formulated for use as pharmaceuticals or cosmetics using well known excipients, although spray formulations, creams, hydrogels and impregnated carrier materials such as dressings, gauzes and bandages are favoured.

Since the compounds of the invention act to inhibit cortisol production they have application in the treatment of diseases caused by increased synthesis of cortisol e.g. Cushing's syndrome.

### BACKGROUND

Extracts of Salvia spp, and a number of tanshinone compounds isolated therefrom, are known to have medicinal properties (see e.g. Journal of Medicinal Plants Research 4, 2813-2820, 29 December Special Review, 2010) and Applicant's own patent publication WO2009050451, teaches the antimicrobial activity of a defined Tanshinone containing extract obtained from a Salvia spp.

About 2% of the general population in the Western world suffer from chronic wounds, causing a significant adverse effect on a patient's Quality of Life. It also creates a significant economic burden, with nearly 2% of the health budgets devoted to the care of chronic wounds and hospitalization (Schreml et al (2010) J Am Acad Dermatol 63, 866-881; Sgonc and Gruber (2012) Gerontology 59, 159-164). Despite this high incidence and economic burden, the outcomes of the management of chronic wounds are far from satisfying and novel therapies are in urgent need to improve patient's Quality of Life and lower health care costs.

Active ingredients with specificity to the pathogenesis of chronic wounds are highly needed. Such active compounds must be able to normalize the “mis-activated” regulatory pathways, and must be devoid of any toxic and allergenic potential.

Applicant has now unexpectedly discovered that a Tanshinone containing extract of Salvia spp, and a number of tanshinone compounds isolated therefrom, particularly tanshinone I and dihydrotanshinone, are potent inhibitors of CYP11B1 and as such can be expected to be useful in treating conditions benefiting from inhibition of cortisol synthesis, such as, the treatment of wounds, particularly chronic wounds, since inhibition of CYP11B1 is beneficial in accelerating wound healing.

The rationale for this therapeutic application derives from the fact that CYP11B1 is the cortisol-producing enzyme expressed in human adrenal glands and skin (FIG. 1) and inhibition of CYP11B1 has been shown to promote wound healing in human skin explants and in vivo in pigs (Vukelic et al (2011) J Biol Chem 286, 10265-10275).

Importantly, the skin of rodents and other lower mammal species is different from human skin with respect to the expression of enzymes involved in cortisol biosynthesis. For example, in mouse, 11beta-HSD1 is upregulated in chronic wounds. Cyp11B1 is neither expressed in unwounded skin nor post-wounding in mice and rats (Tiganescu et al, J Endocrinol 221, 51-61; Dalla Valle et al, J Steroid Biochem Mol Biol 43, 1095-1098).

Significantly the Applicant has determined that certain compounds present in an ethanolic Salvia extract inhibit Cyp11B. Since Cyp11B1 is the critical target in humans they have been able to apply this for use in treating wounds, particularly chronic wounds, and other conditions in humans.

Data reported on e.g. rodents or other lower mammals are not suitable models for predicting wound healing effects in human skin, and would not lead one to conclude they have use in the treatment of e.g. chronic wounds or conditions relating to cortisol production, such as Cushing's syndrome.

In this regard, the most important enzyme in the synthesis of cortisol is CYP11B1. CYP11B1 is the enzyme that converts the inactive glucocorticoid 11-deoxycortisol into highly active cortisol. Expression and activity of CYP11B1 in the human skin is tightly regulated, in particular during wound healing. After wounding, CYP11B1 expression and activity are significantly up-regulated, in particular during the second day, to hold the inflammatory response in check, but return to control values on the third and fourth day after wounding, to prevent glucocorticoid-induced inhibition of keratinocyte proliferation/migration and other important processes that are essential for wound healing (Vukelic et al (2011) J Biol Chem 286, 10265-10275). In chronic wounds, however, expression of CYP11B1 remains permanently elevated (U.S. Pat. No. 8,802,660 B2). Inhibition of the production of cortisol may therefore reverse the deleterious effects of prolonged cortisol exposure in chronic wounds.

Applicant has confirmed CYP11B1 as a target for wound healing using a highly potent CYP11B1 inhibitor that is devoid of 11β-HSD1 inhibitory activity in the same ex vivo human skin wound model as used by Vukelic et al (2011) J Biol Chem 286, 10265-10275). Using this CYP11B1 inhibitor as a chemical probe, they observed a significantly faster healing process, and full wound closure owing to re-epithelialization compared to the vehicle control.

Inhibition of CYP11B1 can therefore be regarded as a novel, highly promising therapy for the treatment of, particularly, chronic wounds. In addition, environmental dryness (also inducing skin barrier dysfunction) significantly increases CYP11B1 expression and activity in a skin equivalent model (Takel et al (2013) Exp Dermatol 22, 662-664).

UV light of short wavelengths (UVB and UVC) is another important environmental stressor that stimulates cortisol and corticosterone synthesis in mammalian skin. The increased synthesis rate was shown to be mediated by an up-regulation of several steroidogenic enzymes in human skin, including CYP11B1 and 11β-hydroxysteroid dehydrogenase (HSD) 1 (Skobowiat et al (2011) Br J Dermatol 168, 595-601; Skobowiat et al (2011) Am J Physiol Endocrinol Metab 301, E484-E493). While this biochemical response is assumed to be protective in young skin, the up-regulation of cortisol production persists in the aged skin and is believed to contribute to the adverse changes in skin morphology and function associated with chronological aging and photo-aging (Tiganescu et al (2011) J Invest Dermatol 131, 30-36). Thus, the inhibition of cortisol synthesis particularly in the aging skin is expected to attenuate adverse age-dependent effects, such as loss of tone and elasticity, increased fragility, increased dryness, decreased thickness, and reduced synthesis of extracellular matrix components such as hyaluronan and collagen (Tiganescu et al (2011) J Invest Dermatol 131, 30-36).

CYP11B1 is also expressed in the gut (Taves et al (2011) Am J Physiol Endocrinol Metabol 301, E11-E24; Fernandez-Marcos et al (2011) Biochim Biophys Acta, 1812, 947-955) and in the oral cavity (Peng et al (2011) PLoS One 6:e23452, data were analyzed using the Oncomine web portal (www.oncomine.org)). Thus, the healing of lesions in epithelial tissues and cavities other than the skin might also be accelerated by the inhibition of CYP11B1 in the respective epithelial cells.

It was furthermore reported that in women with stress-related depression and exhaustion, significantly increased levels of cortisol are present in the crevicular fluid of the gingiva, which are correlated with a higher amount of dental plaque and local inflammation (Johannsen et al (2006) J Periodontology 77,1403-1409). Although it is not known which percentage of the cortisol might also be of systemic origin, the local production might be effectively blocked using CYP11B1 inhibitors, leading to an improvement of the periodontal health.

Prior art identified include the following:

Chinese Journal of Clinical Rehabilitation, Vol 9, No 6, 2005, pages 156-7. This document discloses the use of radix Salviae militiorrhizae on wound healing in rat skin. Rat skin however expresses different enzymes to humans in cortisol production and thus it does not follow that it could be used to treat wounds in humans.

CN102988370 discloses the use of Tanshinone I in the treatment of psoriasis.

CN10282340 discloses the use of Tanshinone IIA in the treatment of psoriasis.

CN12973575 discloses the use of Cryptotanshinone in the treatment of psoriasis.

Journal of the Pharmaceutical society of Japan, vol 131, no 4, 2011 pages 581-586 discloses the use Salvia officinalis L to treat atopic dermatitis in a mouse model.

Chinese Journal of Reparative and Reconstructive Surgery, vol 12, no 4, 1998, pages 205-208 discloses the use of Danshen in rabbits with burned skin. Rabbits are lower mammals and it does not follow that it could be used to treat wounds in humans.

BMC Biotechnology, vol 14, no 1, 2014, page 74:1-10 discloses the use of a transgenic Salvia miltiorrhiza plant expressing human Fibroblast Growth Factor I in wound healing. The data (paragraph bridging pages 3 and 4) shows that the wild type plant extracts did not promote wound healing in rat skin.

Biomaterial, vol 23, 2002, pages 4459-4462, discloses a sustained release implant of herb extract using chitosan. In vivo biodegradation was again tested on rats.

Evidence based Complementary and Alternative medicine, vol 2012, article id 927658 discloses that Tanshinone 11A inhibits growth of keratinocytes, a possible mechanism for its use in the treatment of psoriasis.

### BRIEF SUMMARY OF THE DISCLOSURE

In accordance with a first aspect of the present inventions there is provided a plant extract, derived from a Salvia spp, comprising one or more tanshinone compounds, or one or more tanshinone compounds, including a CYP11B1 inhibitory amount of tanshinone I and/or dihydrotanshinone, for use in the treatment of wounds or Cushing's syndrome.

Most preferably the wounds treated are chronic wounds.

Preferably, though not essentially, the Salvia spp plant extract is one as described and characterised in WO2009050451, which document is incorporated by reference.

An extract exhibiting these beneficial properties may be derived from the root and rhizome of Salvia miltiorrhiza Bunge, a perennial herb from the Labiatae family. In Traditional Chinese Medicine (TCM) it is also referred to as Danshen.

The chemical constituents of Danshen can be divided into two main categories of chemicals:
lipid-soluble, and
water-soluble.

Earlier studies on “active” compounds of Danshen have mainly concentrated on the lipid-soluble compounds, where around 40 compounds have been found so far.

These can be further divided into two groups:
Tanshinones (o-quinone structure), and
Rosiglitazones (o-hydroxy rosiglitazone, paraquinoid structure).

Most of the tanshinone compounds are diterpenes, of which they are mainly diterpene quinones.

Over 40 different compounds have been identified, including, for example: tanshinone, cryptotanshinone, tanshinone IIA, tanshinone IIB, methyltanshinone, hydroxyltanshinone IIA, isotanshinone I, isotanshinone II, isocryptotanshinone, miltirone, L-dihydrotanshinone I, neotanshinone A, B, C, and salviol.

The structures of four of these compounds are illustrated below as they have been specifically identified in significant quantities (by HPLC chromatography) in the extract disclosed in WO2009050451 (FIG. 2 herein).

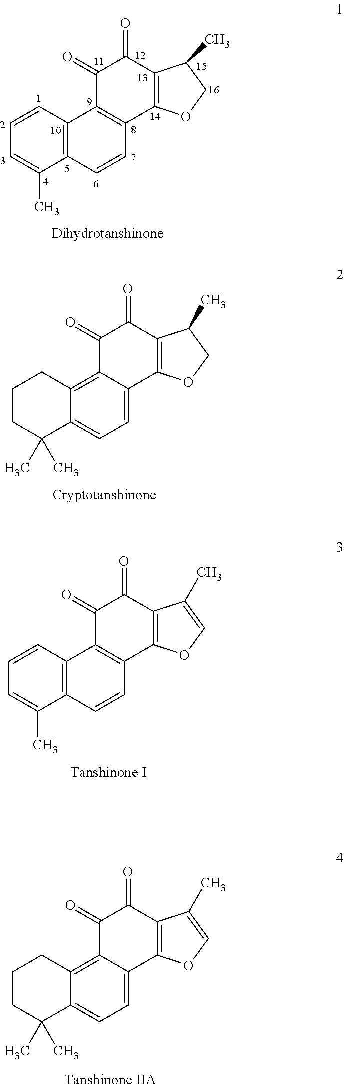 [SMILES: Cc1cccc2c3c(ccc12)C1=C(C(=O)C3=O)[C@@H](C)CO1 ; C[C@H]1COC2=C1C(=O)C(=O)c1c2ccc2c1CCCC2(C)C ; Cc1coc2c1C(=O)C(=O)c1c-2ccc2c(C)cccc12 ; Cc1coc2c1C(=O)C(=O)c1c-2ccc2c1CCCC2(C)C]

Preferably the extract comprises a CYP11B1 inhibitory amount of tanshinone I and/or dihydrotanshinone (more specifically 15,16-dihydrotanshinone I).

In a preferred embodiment the Salvia spp plant extract or one or more tanshinone compounds are for use in the treatment of chronic wounds, with such wounds being prevalent in diabetic or obese patient populations.

Other wounds benefiting from treatment may result from decubitus (bedsores), abrasion, radiation, burns, ulcers or surgical intervention as well as in patient groups where the immune system is compromised.

Another condition benefiting from inhibition of cortisol is Cushing's syndrome.

An exemplary extract is that disclosed in WO2009050451 comprising
Cryptotanshinone,
Dihydrotanshinone,
Tanshinone I, and
Tanshinone IIA,
   characterized in that the above identified tanshinone compounds comprise at least 15%, by weight, of the selectively purified extract and the cryptotanshinone comprises at least 4%, by weight, of the selectively purified extract.

Obviously, alternative Tanshinone containing extracts can be used or preparations comprising or consisting of one or more o-quinones or tanshinones which inhibit CYP11B1 can be used.

According to a second aspect of the present invention there is provided a pharmaceutical or cosmetic comprising or consisting essentially of tanshinone I and/or dihydrotanshinone or an extract of Salvia spp containing same in an amount that will inhibit CYP11B1 by at least 64%, more preferably at least 81% and more preferably still at least 94%.

The skilled person will recognize that it is preferable to maximize the inhibitory effect and typically inhibition of greater than 75% through 80%, 85%, 90% to 95% through 96%, 97%, 98% and 99% to 100%.

An inhibitory amount of therapeutic benefit is one capable of inhibiting the activity of CYP11B1 by at least 60%%, more preferably at least 75% or more.

The pharmaceutical or cosmetic will further comprise one or more excipients.

In a particularly favoured embodiment the active ingredients are carried on a dressing, bandage, gauze or other carrier material.

In another embodiment the active ingredients are incorporated into products for periodontal applications, such as mouthwash, and toothpaste.

According to a third aspect of the invention there is provided a method of treating wounds or Cushing's syndrome comprising providing a patient with a therapeutically effective amount of a salvia spp plant extract, or one or more tanshinone compounds including Tanshinone I and dihydrotanshinone.

Preferably the wounds treated are chronic wounds.

Most preferably the tanshinone compounds are tanshinone I and/or 15,16-dihydrotanshinone I.

The invention is further described, by way of example only, with reference to the following drawings and detailed description.

### BRIEF DESCRIPTION OF THE DRAWINGS

FIG. 1 is a diagram illustrating the biosynthetic pathway of steroid hormones in humans; and

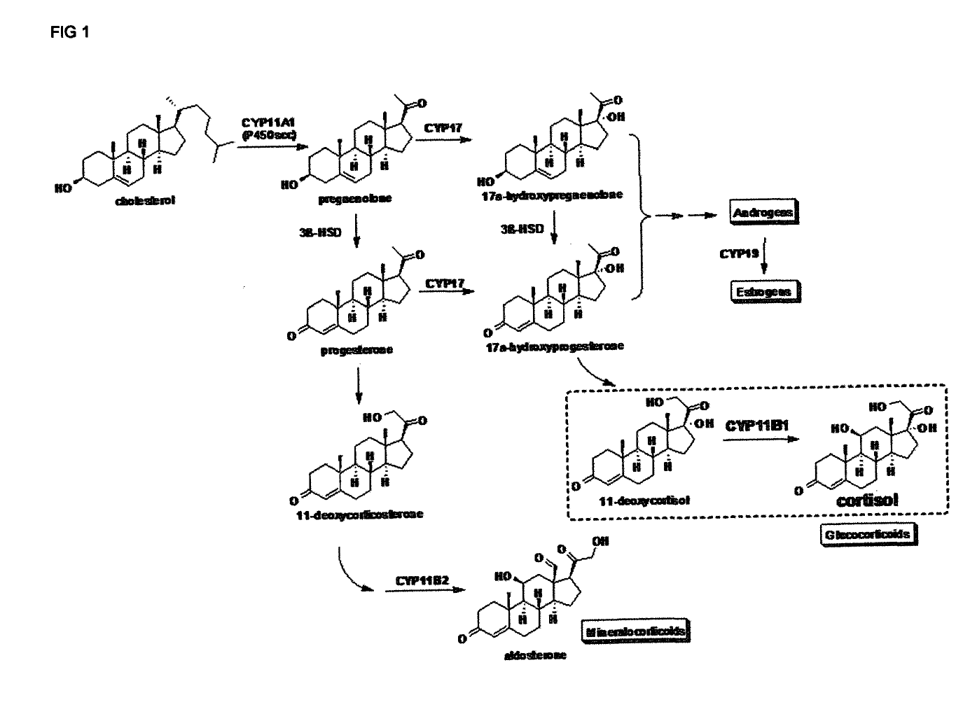

FIG. 2 is an HPLC chromatogram of an extract according to the invention.

### DETAILED DESCRIPTION

Applicant has discovered that Salvia miltiorrhiza Bunge extract (as disclosed in WO2009050451) inhibits CYP11B1 activity in intact cells in a dose dependent manner.

The extract disclosed in WO2009050451 is a selectively purified tanshinone compounds containing extract from the root of a Salvia spp comprising:
Cryptotanshinone,
Dihydrotanshinone,
Tanshinone I, and
Tanshinone IIA,
   characterized in that the above identified tanshinone compounds comprise at least 15%, by weight, of the selectively purified extract, and the cryptotanshinone comprises at least 4%, by weight, of the selectively purified extract.

However, whilst the Salvia spp of WO2009050451 is Salvia miltiorrhiza Bunge, other Salvia spp such as: Salvia apiana, Salvia argentea, Salvia arizonica, Salvia azurea, Salvia camosa, Salvia clevelandii, Salvia coccinea, Salvia divinorum, Salvia dorrii, Salvia farinacea, Salvia forreri, Salvia fulgens, Salvia funerea, Salvia glutinosa, Salvia greggii, Salvia guaranitica, Salvia hispanica, Salvia leucantha, Salvia leucophylla, Salvia libanotica, Salvia longistyla, Salvia lyrata, Salvia mexicana, Salvia officinalis, Salvia patens, Salvia polystachya, Salvia potus, Salvia pratensis, Salvia roemeriana, Salvia sclarea, Salvia spathacea, Salvia splendens, Salvia verticillata, Salvia vitidis may be used to obtain a tanshinone containing extract.

Thus, the extract disclosed in WO2009050451 comprises at least 35%, by weight, of the identified tanshinone compounds with cryptotanshinone comprising at least 15%, by weight, of the selectively purified extract.

Indeed, preferably the identified tanshinone compounds comprised at least 45%, by weight, of the selectively purified extract, and the cryptotanshinone comprised at least 25% by weight, of the selectively purified extract.

In one embodiment the cryptotanshinone comprised at least 20%, more preferably at least 25%, more preferably still at least 40% and maybe as much as 60% of the four identified tanshinone compounds.

Similarly, the tanshinone IIA preferably comprised less than 55% of the four identified tanshinone compounds, more preferably still less than 50%, yet more preferably still less than 40% and might comprise as little as 20% or less of the four identified tanshinone compounds.

Most preferably the extract contains at least 1%, more preferably still at least 2% and more preferably still at least 3% of more of tanshinone I and/or dihydrotanshinone. Indeed the extract may be a highly selective extract containing at least 5%, more preferably at least 10%, through 20%, 30%, 40%, 50%, 60%, 70%, 80% and 90% of the one or more preferred compounds tanshinone I and/ or dihydrotanshinone.

In the embodiment exemplified in Example 1, the selectively purified tanshinone compound containing extract was characterized in that it comprises the four identified tanshinone compounds in an amount of 42.89% (plus or minus 40%, through 30% to 20%):
a cryptotanshinone content of 18.95% (plus or minus 40%, through 30% to 20%),
a dihydrotanshinone content of 3.65% (plus or minus 40%, through 30% to 20%),
a tanshinone I content of 3.82% (plus or minus 40%, through 30% to 20%), and
a tanshinone IIA content of 16.47% (plus or minus 40%, through 30% to 20%).

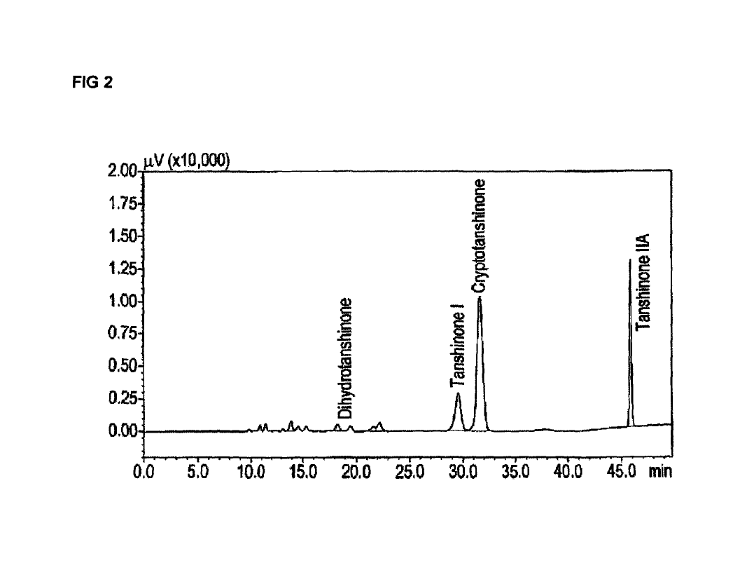

This selectively purified tanshinone compound containing extract was characterized in that it has an HPLC fingerprint substantially as illustrated in FIG. 2 with characteristic peaks as indicated.

However, it will be apparent from Example 3 (herein) that, whilst the extract is a potent CYP111B1 inhibitor, two of the lesser present Tanshinones, 15,16-dihydrotanshinone and tanshinone I are significantly more active than the major tanshinones present, cryptotanshinone and tanshinone IIA, and consequently it may be preferred to use alternative extracts with higher contents of one or more of the 15,16-dihydrotanshinone or tanshinone I, or indeed use the isolated compounds (or synthetically manufactured compounds or derivatives) either alone or together with one another.

Similarly, whilst the extract described above was prepared from the root of a Salvia spp comprising the steps of:
soaking raw material in strong ethanol, for a time sufficient to solublize the tanshinone compounds,
extracting the tanshinone compounds containing fraction using a percolation method, and
concentrating the desired fraction under vacuum, and recovering the ethanol
   it may be preferable to modify the process to concentrate or preferentially select the 15,16-dihydrotanshinone or tanshinone I.

Thus, alternative methodology to that disclosed in WO2009050451, namely utilising a first purification step comprising:
a. dissolving the extract in sufficient water,
b. allowing the desired fraction to precipitate out,
c. discarding the aqueous solution, and
d. collecting the precipitate.
   might be used.

Similarly, whilst WO2009050451 discloses a second purification comprising
e. a separation on a macroporous resin column (AB 8 macroporous resin column, manufactured by Lioayuan New Materials Ltd, or another suitable column) alternative methodology with specificity to the preferred compounds may be desired.

Details of the experiments supporting the claims are set out below:

### EXAMPLE 1

### 1.1. Preparation of the Extract Solutions

Applicant dissolved ˜10 mg of the Salvia m. Bunge extract (as disclosed in WO2009050451) in the required volume of 100% ethanol or 100% DMSO to obtain a 1% (w/v) extract solution. They tested 5 μL of this solution in a 500 μL assay incubation volume (final ethanol or DMSO conc. of 1%). From this 1% Salvia m. Bunge extract solution, they also prepared a 1:10 and 1:100 dilution in 100% ethanol or 100% DMSO. From these solutions, they tested 5 μL in a 500 μL assay incubation volume.

1.2. CYP11B1 Assay

The V79MZh11B1 cell line, expressing recombinant human CYP11B1, was cultured in Dulbecco's modified Eagle (DME, Sigma) medium supplemented with 5% fetal calf serum (FCS; Sigma), penicillin G (100 U/ml), streptomycin (100 μg/ml), glutamine (2 mM) and sodium pyruvate (1 mM) at 37° C. in 5% CO₂ in air. Cells were placed on 24-well cell culture plates (8×10⁵ cells per well) and cultured in 1 ml DME medium per well until confluence. On the day of testing, DME medium was removed and 450 μl of fresh DMEM, containing 5 μl of the extract solution in 100% ethanol or 100% DMSO, was added to each well. There was no significant difference in CYP11B1 inhibition between DMSO or ethanol as solvent. Control wells (receiving vehicle or ketoconazole (final concentration of 50 nM) as reference compound to validate each experiment) were treated in the same way without extract solution. After 60 min at 37° C. in the CO₂ incubator, the reaction was started by the addition of 50 μl of DMEM containing 100 nM of 11-deoxycorticosterone (plus 0.15 μCi of [1,2-³H] 11-deoxycorticosterone) as substrate. All measurements were in duplicate. After 25 min, the enzyme reaction was stopped by extracting the supernatant with ethyl acetate. Samples were centrifuged (10,000×g, 10 min), and the upper phase was pipetted into fresh cups. The ethylacetate solvent was evaporated and the residue was dissolved in 40 μl of methanol and analyzed by HPLC. The following formulas were used to determine the level of conversion and percentage of enzyme inhibition.

$$CYP 11 B 1$$

$$Conversion = 100 \% * \frac{area ( corticosterone )}{area ( 11 - deoxycorticosterone + corticosterone )}$$

$$\% Inhibition = 100 \% - \frac{conversion\_with \_inhibitor}{conversion\_without \_inhibitor}$$

### Results

The results are presented in Table 1 which shows the inhibition of CYP11B1 activity by Salvia m. Bunge extract in V79MZh11B1 cells. The extract solutions were freshly made from dry extract at the day of the experiment.

TABLE 1
Table 1. Determination of CYP11B1 inhibition by Salvia m.
Bunge extract. The extract solutions were freshly made from
dry extract at the day of the experiment in either 100% ethanol or
100% DMSO. N denotes the number of independent experiments.

| | | % | |
|---|---|---|---|
| | Final extract conc. | CYP11B1 inhibition | Number of |
| | in CYP11B1 assay | (mean ± SD) | experiments |
| Ethanol | 0.0001% | 22.6 ± 9.9% | N = 5 |
| | 0.001% | 79.1% | N = 1 |
| | 0.01% | 95.6 ± 4.4% | N = 6 |
| DMSO | 0.0001% | 16.8 ± 1.5% | N = 2 |
| | 0.001% | 83.1 ± 11.4% | N = 2 |
| | 0.01% | 100.0% | N = 1 |

As shown, the Salvia m. Bunge extract prepared in 100% ethanol and 100% DMSO at a final concentration of 0.01% inhibited human CYP11B1 by 95.6% and 100.0%, respectively. From the 1% extract solutions, Applicant made a 1:10 dilution in 100% ethanol or 100% DMSO, respectively. From these latter solutions, they tested 5 μL in 500 μL assay volume. These extract solution (final extract concentration of 0.001% in the assay) inhibited human CYP11B1 by 79.1% and 83.1%, respectively. From the 1% extract solutions, Applicant made also 1:100 dilution in 100% ethanol or 100% DMSO, respectively. From the latter solution, they tested 5 μL in 500 μL assay volume. This extract solution (final extract concentration of 0.0001% in the assay) inhibited human CYP11B1 by 22.6% and 16.8%, respectively.

The conclusion from these experiments was that Salvia m. Bunge extract inhibits CYP11B1 at a dilution of 0.0001%, 0.001% and 0.01%.

In order to check that the inhibition was not due to toxicity, a MTT [3-(4,5-dimethylthiazol-2-yl)-2,5-diphenyl-tetrazolium bromide] cellular viability assay was performed in the same cell line under the incubation conditions used in the CYP11B1 screening assay as set out in Example 2 below:

### EXAMPLE 2

### MTT Cellular Viability Assay

V79MZh11B1 cells were cultured on 24-well cell culture plates (8×10⁵ cells per well) in 1 ml DME medium until confluence. On the day of testing, DME medium was removed and 450 μl of fresh DME medium with 5% FCS, containing 5 μl of the Salvia m. Bunge extract solution in 100% ethanol, was added to each well. Ethanol (1%) and Triton® X-100 (0.0006%) were used as vehicle and positive control (all final concentrations), respectively. All measurements were in quadruplicate. After 60 min at 37° C. in a 5% CO₂, 50 μl of fresh DME medium (+5% FCS) was added to each well. After 25 min, medium was replaced by 500 μl fresh DME medium (+5% FCS) to which 25 μl of MTT solution (5 mg per ml PBS, pH 7.2) was added immediately. After 30 min, all medium was removed and the cells were lysed in 250 μl of 0.5% acetic acid (v/v), 10% SDS (w/v) in DMSO. Absorbance of formazan was measured spectrophotometrically at 570 nm wavelength

Results

Determination of the Effect of 0.01% and 0.0001% Salvia m. Bunge Extract Solution on Cellular Viability of V79MZh11B1 Cells

The effect of Salvia m. Bunge extract on cellular viability of V79MZh11B1 cells under the (pre-) incubation conditions used in the CYP11B1 screening assay was determined. As shown in Table 2, the 0.01% and 0.0001% Salvia m. Bunge extract solutions had no effect on the conversion of MTT into formazan (whereas the positive control, Triton® X-100, did almost fully block formazan formation). Therefore, it was concluded that the inhibitory effect of Salvia m. Bunge extract on CYP11B1 is not caused by a cytotoxic effect.

TABLE 2
Table 2. Lack of effect of Salvia m. Bunge extract on viability
of V79MZh11B1 cells in the MTT toxicity assay. The extract
solutions were freshly made from dry extract at the day of each
experiment. MTT conversion into formazan, in the presence of
1% ethanol only, was set at 100% (data are mean ± SD).

| | | Final conc. | |
|---|---|---|---|
| Experiment nr. | Treatment | in assay | Cell viability |
| Experiment 1 | Salvia m. bunge extract | 0.01% | 111 ± 2% |
| | Salvia m. bunge extract | 0.0001% | 101 ± 1% |
| | Ethanol (vehicle) | 1% | 100 ± 3% |
| | Triton X-100 (pos. control) | 0.0006% | 3 ± 1% |
| Experiment 2 | Salvia m. bunge extract | 0.01% | 124 ± 7% |
| | Salvia m. bunge extract | 0.0001% | 122 ± 11% |
| | Ethanol (vehicle) | 1% | 100 ± 14% |
| | Triton X-100 (pos. control) | 0.0006% | 3 ± 1% |

### EXAMPLE 3

Given the activity of the extract the Applicant looked at the activity of some of the tanshinones using the methodology described in Example 1.

The tanshinones tested in V79MZh11B1 cells were:
tanshinone IIA,
tanshinone I,
dihydrotanshione I, and
cryptotanshinone.

The results are shown in Table 3 below:

TABLE 3
Table 3. CYP11B1 inhibitory effect of tanshinone IIA,
tanshinone I, dihydrotanshione I and cryptotanshinone.
The results are mean ± SD of 2 independent experiments.

| Tanshinone IIA | % CYP11B1 | Tanshinone I | % CYP11B1 |
|---|---|---|---|
| Concentration | Inhibition | Concentration | Inhibition |
| 1 μM | 5.9 ± 0.2 | 1 μM | 19.1 ± 7.9 |
| 10 μM | 16.1 ± 13.5 | 10 μM | 63.7 ± 4.5 |
| 100 μM | 29.4 ± 0.4 | 100 μM | 81.1 ± 6.9 |
| Dihydrotanshinone | % CYP11B1 | Cryptotanshinone | % CYP11B1 |
| Concentration | Inhibition | Concentration | Inhibition |
| 1 μM | 43.3 ± 14.7 | 1 μM | 6.4 ± 1.6 |
| 10 μM | 93.6 ± 9.1 | 10 μM | 15.4 ± 10.3 |
| 100 μM | 100.0 ± 0.0 | 100 μM | 59.9 ± 22.0 |

It will be apparent from the results that each of the tanshinones exhibited inhibitory activity, with the two most effective ones being dihydrotanshinone (94% inhibition at 10 μM) and Tanshinone I (64%-inhibition at 10 μM).

This in itself was unexpected, since these two compounds are present in lower amounts in the extract disclosed in WO2009050451 (respectively 3.65% and 3.82%) than cryptotanshinone and Tanshinone Ila (18.95% and 16.47% respectively).

Looking at the structures, it is possible that the enhanced activity of dihydrotanshinone (94% inhibition at 10 μM) and Tanshinone I (64% inhibition at 10 μM) might be attributed to the presence of a methyl (as opposed to a dimethyl) grouping at the C4 position.

Given the activity of these structurally related compounds, it is likely that other members of the Tanshinone family of compounds (or derivatives thereof) might be expected to exhibit similar (or better) CYP11B1 inhibitory activity.

### EXAMPLE 4

Test for Thermal Stability at 70° C., 80° C. and 90° C. of Salvia m. Bunge Extract

In general, wound plaster constituents are frequently briefly held at elevated temperatures (70° C.-90° C.) to reduce the number of potential residual germs. It is therefore important that the CYP11B1 inhibitory activity of the extract/tanshinones should be stable at these elevated temperatures if they are to be used in situations where plaster is placed around a wound postoperatively.

The CYP11B1 inhibitory potency of the Salvia m. Bunge extract was determined after 5 min and 15 min of treatment at 70° C., 80° C. or 90° C. Salvia m. Bunge extract was dissolved in a 100% DMSO solution (at a concentration of either 0.05% and 0.025%) and incubated at 70° C., 80° C. and 90° C. for either 5 or 15 min, followed by testing in the CYP11B1 assay at a final concentration of 0.0005% and 0.00025%. These concentrations were chosen around the IC50 of the extract which inhibits CYP11B1.

The results are illustrated in Table 4 below which shows the thermal stability of the Salvia m. Bunge extract. Data are the mean±SD of either 4 (control) or 2 measurements:

TABLE 4
Table 4. Thermal stability of the Salvia m. Bunge extract. Data
are the mean ± SD of either 4 (control) or 2 measurements.

| | |
|---|---|
| Pretreatment of | % Cyp11b1 inhibition |

| 0.05% or 0.025% | 0.0005% | 0.00025% |
|---|---|---|
| solution | dilution | dilution |
| Control (25° C.) | 42.2 ± 2.5% | 23.6 ± 4.2% |
| 70° C., 5 min | 43.3 ± 0.6% | 25.2 ± 3.8% |
| 80° C., 5 min | 43.2 ± 1.8% | 25.0 ± 13.4% |
| 90° C., 5 min | 40.6 ± 5.6% | 27.3 ± 3.4% |
| 70° C., 15 min | 45.8 ± 0.6% | 24.1 ± 1.9% |
| 80° C., 15 min | 42.7 ± 7.4% | 30.2 ± 2.6% |
| 90° C., 15 min | 44.4 ± 3.6% | 29.5 ± 2.4% |

No effect on CYP11B1 inhibitory activity was seen in any pretreatment at 70° C., 80° C. and 90° C. in comparison to control values (which were determined with the Salvia m. Bunge extract pretreated at 25° C. at a concentration of 0.05% and 0.025% for 15 min). Accordingly, these extracts/compounds may be used in situations where the wound is set in plaster.

### EXAMPLE 5

Test for Allergenicity of the Salvia m. Bunge Extract.

Potential allergenicity of the extract was tested on intact human skin (inside of the upper arm) in three volunteers at a Salvia m. Bunge concentration of 0.5% (weight/volume) in 100% Vaseline®. No sign of allergenicity (i.e. change in skin colour or texture) was seen in any individual during the five days of skin exposure.

From the above Examples it can be concluded that a plant extract, derived from a Salvia spp, comprising one or more tanshinone compounds, or said one or more tanshinone compounds, look promising candidates for use in the treatment of wounds or other conditions benefiting from inhibition of cortisol synthesis.

In addition, Applicant has identified that the two particularly active constituents are rather lipophilic (the log calculated using ACD/log P GALAS is 3.57 for dihydrotanshinone I), suggesting a good penetration to the epidermis which is essential to an efficient inhibition of the epidermally expressed target enzymes.

1. A plant extract, derived from a Salvia spp, comprising one of:
at least one tanshinone compound; or
at least one tanshinone compound, including a CYP11B1 inhibitory amount of at least one of tanshinone I and dihydrotanshinone,
wherein the plant extract is for use in the treatment of a wound or Cushing's syndrome.

2. A plant extract as claimed in claim 1, wherein the wound is a chronic wound.

3. A plant extract as claimed in claim 2, wherein the chronic wound is associated with diabetes.

4. A plant extract as claimed in claim 2, wherein the chronic wound is a venous or arterial ulcer.

5. A plant extract as claimed in claim 2, wherein the chronic wound is associated with prolonged pressure.

6. A plant extract as claimed in claim 2, wherein the chronic wound is associated with radiation burns.

7. A plant extract as claimed in claim 1, wherein the plant extract is for use in the treatment of Cushing's syndrome.

8. A plant extract as claimed in claim 1, comprising cryptotanshinone, dihydrotanshinone, tanshinone I, and tanshinone IIA, wherein the tanshinone I and the tanshinone IIA comprise at least 15%, by weight, of the plant extract, and the cryptotanshinon comprises at least 4%, by weight, of the plant extract.

9. A pharmaceutical or cosmetic comprising one of:
at least one of tanshinone I and dihydrotanshinone, or
an extract of Salvia spp containing at least one of tanshinone I and dihydrotanshinone,
wherein the at least one of tanshinone I and dihydrotanshinone is in an amount that will inhibit CYP11B1 by at least 64%.

10. A pharmaceutical or cosmetic as claimed in claim 9, further comprising at least one excipient.

11. A pharmaceutical or cosmetic as claimed in claim 9, further comprising a carrier material.

12. A pharmaceutical or cosmetic as claimed in claim 9, wherein the pharmaceutical or cosmetic is used for periodontal applications.

13. A method of treating a wound or Cushing's syndrome, the method comprising providing a patient with one of:
a therapeutically effective amount of a Salvia spp plant extract, or
at least one tanshinone compound including a CYP11B1 inhibitory amount of at least one of tanshinone I and dihydrotanshinone.

14. A method as claimed in claim 13, wherein the wound is a chronic wound.

15. A pharmaceutical or cosmetic as claimed in claim 9, wherein the at least one of tanshinone I and dihydrotanshinone is in an amount that will inhibit CYP11B1 by at least 81%.

16. A pharmaceutical or cosmetic as claimed in claim 15, wherein the at least one of tanshinone I and dihydrotanshinone is in an amount that will inhibit CYP11B1 by at least 94%.

17. A pharmaceutical or cosmetic as claimed in claim 11, wherein the carrier material is one of a dressing and a bandage.

18. A pharmaceutical or cosmetic as claimed in claim 10, wherein the pharmaceutical or cosmetic is used for periodontal applications.

19. A pharmaceutical or cosmetic as claimed in claim 18, wherein the periodontal applications include at least one of mouthwash and toothpaste.

20. A pharmaceutical or cosmetic as claimed in claim 12, wherein the periodontal applications include at least one of mouthwash and toothpaste.
